(19) United States
(12) Patent Application Publication
(10) Pub. No.: US 2018/0360924 A1
(43) Pub. Date: Dec. 20, 2018

(54) TELOMERE EXTENSION AND ANTI-INFLAMMATORY AGENTS FOR CELL REGENERATION

(71) Applicant: THE METHODIST HOSPITAL SYSTEM, Houston, TX (US)

(72) Inventors: Mir Timo Zadegh NAZARI-SHAFTI, Luebeck (DE); John P. COOKE, Houston, TX (US)

(21) Appl. No.: 15/779,257

(22) PCT Filed: Nov. 23, 2016
(86) PCT No.: PCT/US2016/063545

§ 371 Date: May 25, 2018

Related U.S. Application Data
(60) Provisional application No. 62/260,020, filed on Nov. 25, 2015.

(51) Int. Cl. A61K 38/45; A61K 38/16; A61K 31/519; A61K 31/575; A61K 38/20; A61K 9/127; A61K 35/32; A61K 45/06; A61K 31/405; A61K 31/192; A61K 31/415; A61K 31/573; A61K 31/436; A61K 31/616; A61K 31/167

(52) U.S. Cl. CPC A61K 38/45; A61K 31/167; A61K 38/162; A61K 31/519; A61K 31/575; A61K 38/2066; A61K 9/127; A61K 35/32; A61K 45/06; A61K 31/405; A61K 31/192; A61K 31/415; A61K 31/573; A61K 31/436; A61K 31/616; C12Y 207/07049

(57) ABSTRACT

Disclosed is a method for rejuvenating cells, such as chondrocytes, that involves contacting the cell with a composition comprising a synthetic ribonucleic acid comprising at least one modified nucleoside encoding a telomerase reverse transcriptase, and a composition comprising an anti-inflammatory agent, in amounts effective to extend at least one telomere in the cell.

### CROSS-REFERENCE TO RELATED APPLICATIONS

This application claims benefit of U.S. Provisional Application No. 62/260,020, filed Nov. 25, 2015, which is hereby incorporated herein by reference in its entirety.

### STATEMENT REGARDING FEDERALLY SPONSORED RESEARCH OR DEVELOPMENT

This invention was made with Government Support under Grant No. U01HL100397 awarded by the National Institutes of Health. The Government has certain rights in the invention.

### BACKGROUND

Telomeres comprise repetitive DNA sequences at the ends of linear chromosomes that, when sufficiently long, allow each chromosome end to form a loop that protects the ends from acting as double-stranded or single-stranded DNA breaks. Telomeres shorten with cell replication (due to the end-replication problem), and/or due to oxidative damage and other stresses, eventually leading to critically short telomeres unable to form the protective loop, leading to exposure of the chromosome ends, chromosome-chromosome fusions, DNA damage responses, and cellular senescence, apoptosis, or malignancy.

The enzyme complex telomerase extends telomeres and comprises two essential components: the telomerase reverse transcriptase (TERT), and an RNA component known as telomerase RNA component (TERC). Other components of the telomerase complex include the proteins TCAB1, Dyskerin, Gar1, Nhp2, Nop10, and RHAU. TERT is a limiting component of the telomerase complex, and thus treatments that increase TERT can increase telomerase activity. Telomerase activity is typically measured using the telomeric repeat amplification protocol (TRAP) assay, which quantifies the ability of a cell lysate or other sample to extend a synthetic telomere-like DNA sequence.

As would be expected due to the importance of telomere length maintenance in preventing cellular senescence and apoptosis and resulting cellular dysfunction, genetic mutations of TERT and TERC are linked to fatal inherited diseases of inadequate telomere maintenance, including forms of idiopathic pulmonary fibrosis, dyskeratosis congenita, and aplastic anemia. The effects of premature cellular senescence and apoptosis due to short telomeres in these diseases are devastating in themselves, and may be compounded by increased risk of cancer. In addition to abundant correlative data linking short telomeres to cancer, aplastic anemia provides some of the first direct evidence that critically short telomeres and resulting chromosomal instability predispose cells to malignant transformation in humans. There is evidence that short telomeres make the difference between fatal and non-fatal muscular dystrophy. In children with Progeria and other disorders characterized by accelerated aging, telomeres are shorter and associated with more rapid cellular senescence. These children are particularly afflicted by accelerated aging of the cardiovascular system leading to heart attack, stroke and death in the teen years. Of note, telomere extension can reverse vascular cell senescence. Since vascular cell senescence is associated with atherosclerosis, hypertension, and heart disease, telomere extension can also be useful in these disorders. In addition to being implicated in these and other diseases, short telomeres also limit cell amplification for cell therapies and bioengineering applications. In addition, there are a number of diseases of pets and livestock that would benefit from telomere extension, such as chronic renal disease in cats, where shortened telomeres may contribute to the disease.

Human cells with little or no telomerase activity have been transfected with vectors encoding human TERT (hTERT). The transfected cells were found to express telomerase, to display elongated telomeres, to divide vigorously, and to display reduced senescence compared to cells lacking telomerase, but the genomic modification resulting from this treatment increases the risk and limits the utility of the approach.

A limited capacity to replicate is one of the defining characteristics of most normal cells. An end-point of this limited replicative process is senescence, an arrested state in which the cell remains viable but no longer divides. Senescent cells are often characterized by an altered pattern of gene expression, altered morphology, and reduced or abrogated ability to perform their normal functions.

The shortening of telomeres plays a direct role in cellular senescence in animal tissues during aging. Furthermore, there is accumulating evidence implicating short telomeres in a variety of diseases, including those described above. The prospect of preventing disease by telomere extension motivates a need for safe and effective treatments to extend telomeres in animal cells in vivo and/or in vitro. Further, there is a need to safely and rapidly extend telomeres in cells for use in cell therapy, cell and tissue engineering, and regenerative medicine.

At the same time, however, there is a danger in the constitutive activation of telomerase activity. Indeed for cell therapy applications, avoiding the risk of cell immortalization is of paramount importance. To this end, transient, rather than constitutive, telomerase activity may be advantageous for safety, especially if the elevated telomerase activity is not only brief but extends telomeres rapidly enough that the treatment does not need to be repeated continuously. Current methods of extending telomeres include viral delivery of TERT under an inducible promoter, delivery of TERT using vectors based on adenovirus and adeno-associated virus, and small molecule activators of telomerase. These methods risk either insertional mutagenesis, continual elevation of telomerase activity, or both.

Thus, there is strong motivation to develop a therapy that safely extends telomeres to potentially prevent, delay, ameliorate, or treat these and other conditions and diseases, to do the same for the gradual decline in physical form and function and mental function that accompanies chronological aging, and to enable cell therapies and regenerative medicine.

Chondrocytes needed for restoring joint cartilage do not replicate well, particularly in older patients. While RNA encoding TERT (“TERT RNA”), including mmRNA hTERT, has been shown to extend telomeres, decrease markers of senescence, and enhance replicative capacity in human fibroblasts, endothelial cells and myoblasts, mmRNA hTERT surprisingly impairs replicative capacity, increases senescence markers, and does not increase telomere length in human chondrocytes. Methods are therefore needed to rejuvenate (as defined by decreased senescence markers, increased replicative capacity, enhanced function, and/or increased telomere length) chondrocytes and/or other cells that are not rejuvenated by TERT RNA, including mmRNA hTERT, alone. In addition, these methods may also be useful to further enhance the rejuvenating effect of TERT RNA, including mmRNA hTERT, on cells that are responsive to RNA TERT alone.

### SUMMARY

Disclosed is a method for causing or assisting with the rejuvenation of an RNA immune-responsive cell, comprising contacting the RNA immune-responsive cell with a composition comprising a synthetic RNA encoding telomerase reverse transcriptase (tert rna), and a composition comprising an anti-inflammatory agent, in amounts effective to cause or assist with the rejuvenation of the RNA immune-reactive cell. In some embodiments, the method will extend at least one telomere in the RNA immune-responsive cell. In some embodiments, the RNA TERT and anti-inflammatory agent are in the same composition.

The RNA immune-responsive cell can be any cell where innate immunity is excessively activated in response to contact with a TERT RNA, including hTERT mmRNA, and as a consequence the optimal rejuvenating benefit of the RNA encoding TERT is not obtained. This immunity is therefore inhibited by the disclosed anti-inflammatory agent. For example, in some embodiments, the RNA immune-responsive cell is a cell that has upregulated RANTES expression when contacted with the synthetic RNA encoding a telomerase reverse transcriptase. Therefore, RANTES expression can be used as an assay to identify cells that can be caused to be rejuvenated or assisted in their rejuvenation by the disclosed methods. Likewise, this assay can be used to identify anti-inflammatory agents that can be used to inhibit the excessive innate immune response of the RNA immune-responsive cell. In some embodiments, another established marker of innate immune activation may be utilized, such as interleukin-1 or interferons Types I, II, or III. In some embodiments, the cell comprises a chondrocyte. For example, the chondrocyte can be obtained from a subject with cartilage degeneration prior to the contacting step. In some embodiments, the RNA immune-responsive cell comprises a mesenchymal stem cell (MSC). In some embodiments, the RNA immune-responsive cell is a vascular cell. In some embodiments, the animal cell is any cell in which the rejuvenating effect of RNA encoding TERT is enhanced by an anti-inflammatory agent.

In some embodiments, the anti-inflammatory agent comprises an interferon antagonist, such as the B18R protein. In some embodiments, the anti-inflammatory agent comprises an NFκB antagonist, such as the RelA/NFkB p65 [p Ser529, p Ser536] inhibitor peptide.

In some embodiments, the anti-inflammatory agent comprises a Jak-Stat inhibitor, such as Tofacitinib, Curcurbitacin, Colchicine, or a combination thereof. In some embodiments, the anti-inflammatory agent comprises a non-steroidal anti-inflammatory drug (NSAID), such as Indomethacin, Ibuprofen, or Celecoxib. In some embodiments, the anti-inflammatory agent comprises a steroid, such as celastrol, dexamethasone, or prednisone. In some embodiments, the anti-inflammatory agent comprises an immunosuppressant, such as rapamycin, everolimus, or a combination thereof. In some embodiments, the anti-inflammatory agent comprises an anti-inflammatory cytokine, such as IL-10. In some embodiments, the anti-inflammatory agent comprises an analgesic, such as aspirin, acetaminophen, or a combination thereof.

In some embodiments, the disclosed method further comprises measuring telomerase activity or length in the RNA immune-responsive cell prior to the contacting step. In some cases, the RNA immune-responsive cell has at least one shortened telomere prior to the contacting step. In some embodiments, the average telomere length in the RNA immune-responsive cell is increased by at least 0.1 kb in response to the disclosed compositions.

In some embodiments, the telomerase reverse transcriptase is a mammalian, avian, reptilian, or fish telomerase reverse transcriptase or a variant that retains telomerase catalytic activity, including a chimeric TERT that includes sequence from different species. In some embodiments, the telomerase reverse transcriptase is a human telomerase reverse transcriptase. In some embodiments, the ribonucleic acid codes for a polypeptide with at least 95% sequence identity to a human telomerase reverse transcriptase.

In some embodiments, the codon sequence of the hTERT mRNA is optimized by selecting codons that enhance RNA stability and translation for a specific mammalian cell.

In some embodiments, the ribonucleic acid comprises a 5′ cap, a 5′ untranslated region, a 3′ untranslated region, and a poly-A tail. The 5′ cap may be non-immunogenic and the 5′ cap may have been treated with phosphatase.

In some embodiments, the poly-A tail increases stability of the ribonucleic acid.

In some embodiments, the 5′ untranslated region or the 3′ untranslated region comprise a sequence from a stable mRNA or an mRNA that is efficiently translated, or they both comprise a sequence from a stable mRNA or an mRNA that is efficiently translated.

In some embodiments, the 5′ cap, the 5′ untranslated region, or the 3′ untranslated region stabilizes the ribonucleic acid, increases the rate of translation of the ribonucleic acid, or modulates the immunogenicity of the ribonucleic acid.

In some embodiments, the ribonucleic acid is a purified synthetic ribonucleic acid. In some embodiments, the synthetic ribonucleic acid is purified to remove immunogenic components.

In some embodiments, the RNA TERT is replaced by a viral or plasmid vector comprising the TERT sequence.

In some embodiments, the RNA TERT is replaced by a small molecule that increases telomerase activity.

In some embodiments, contacting the RNA immune-responsive cell with the composition comprising the synthetic ribonucleic acid involves electroporation or other physical techniques such as the cell squeezer. In some embodiments, the composition comprising the synthetic ribonucleic acid further comprises a delivery vehicle, such as a transfection agent. In some embodiments, the delivery vehicle is an exosome, a lipid nanoparticle, a polymeric nanoparticle, a natural or artificial lipoprotein particle, a cationic lipid, a protein, a protein-nucleic acid complex, a liposome, a virosome, or a polymer. In some embodiments, the delivery vehicle is a liposome comprising DOTAP and cholesterol in a 1:1 molar ratio. In some embodiments, the delivery vehicle is a liposome that comprises protamine or another protein that contains multiple lysine and/or arginine residues to increase the positive charge of the protein. In some embodiments, the delivery vehicle is non-immunogenic. In some embodiments, the delivery vehicle is partly immunogenic. In particular, under some circumstances, it may be desirable for the vehicle to retain some immunogenicity.

In some embodiments, the RNA immune-responsive cell has at least one shortened telomere prior to the administering step. In some embodiments, the RNA immune-responsive cell is from or in a subject suffering from or at risk of an age-related illness, an age-related condition, or an age-related decline in function or appearance. In some embodiments, the RNA immune-responsive cell is from or in a subject suffering from or at risk of cancer, heart disease, stroke, diabetes, diabetic ulcers, Alzheimer's disease, osteoporosis, a decline in physical ability or appearance, physical trauma or chronic physical stress, psychological trauma or chronic psychological stress, reduced immune function, immunosenescence, or macular degeneration. In some embodiments, the RNA immune-responsive cell is a somatic cell of endodermal, mesodermal, or ectodermal lineage. In some embodiments, the RNA immune-responsive cell is a transdifferentiated cell or a cell used to produce a transdifferentiated cell.

In some embodiments, the RNA immune-responsive cell is an isolated cell, and the administering step lasts no longer than 48 hours. In other embodiments, the RNA immune-responsive cell is an isolated cell, and the administering step lasts at least 2 hours. In some embodiments, the RNA immune-responsive cell is an isolated cell, and the administering step is performed no more than four times. In other embodiments, the cell is an isolated cell, and the administering step is performed at least two times. In some embodiments, the RNA immune-responsive cell is an isolated cell, and the method further comprises the step of measuring telomerase activity in the cell. In specific embodiments, the administering step increases telomerase activity in the cell, and in even more specific embodiments, the telomerase activity is transiently increased by at least 5%. In other specific embodiments, the half-life of increased telomerase activity is no longer than 48 hours.

In some embodiments, the cell is an isolated cell, and the method further comprises the step of measuring population doubling capacity in the cell. In specific embodiments, the population doubling capacity increases, in some cases by at least one population doubling.

In some embodiments, the RNA TERT is administered in vivo to a mammal by a topical, oral, intravenous, intra-arterial, intraperitoneal, intrathecal, rectal, urethral or inhaled route, using an appropriate vehicle that will maintain stability and enhance delivery to the desired tissue or organ.

Also disclosed are kits for extending telomeres in an animal cell, the kits comprising any of the above compounds or compositions and instructions for using the compound or composition to extend telomeres. In some embodiments, the kits further comprise packaging materials. In some embodiments, the packaging materials are air-tight. In some embodiments, the packaging materials comprise a metal foil container. In some embodiments, the kits further comprise a desiccant, a culture medium, or an RNase inhibitor. In some embodiments, the composition is sterile. In some embodiments, the kit comprises a disclosed composition, instructions for using the composition to extend telomeres, and a telomerase RNA component, a delivery vehicle, an anti-inflammatory agent, or any combination thereof.

The disclosed method can further involve administering a plurality of the rejuvenated RNA immune-responsive cells to a subject in need thereof.

Also disclosed is a method for regenerating cartilage in a subject, comprising administering to the subject a plurality of rejuvenated chondrocyte produced by the disclosed methods.

Also disclosed is a method for regenerating cartilage in a subject, comprising administering to the subject a composition comprising a synthetic ribonucleic acid comprising at least one RNA encoding a telomerase reverse transcriptase, and a composition comprising an interferon antagonist, in amounts effective to extend at least one telomere in chondrocytes within the cartilage. In some embodiments, the method comprises administering to the subject a composition comprising the synthetic RNA TERTand the interferon antagonist. For example, in some embodiments, the composition is administered within the joint capsule of the subject.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A and 1B are bar graphs showing chondrocyte doubling times (in hours) after no treatment, treatment with lipofectamine, treatment with hTERT CI, or treatment with hTERT WT. Results are shown after transfection (FIG. 1A, n=6) or 1 passage after transfection (FIG. 1B, n=3).

FIG. 2 is a bar graph showing gene array results for RANTES, HLA-B, IFNB1, IL1A, IL8, MX1, RAG1, TLR1, NFκB1, and NFκB1A 48 hours after second transfection with lipofectamine, hTERT CI, or hTERT WT.

FIGS. 3A to 3D are images of chondrocytes after transfection with hTERT (FIG. 3A), lipofectamine (FIG. 3B), hTERT+B18R (FIG. 3C), or hTERT+p65i (FIG. 3D).

FIGS. 4A to 4D are bar graphs showing total cell number after two transfections (FIGS. 1A & 1B) and cell doubling time (FIGS. 1C & 1D, hours) of chondrocytes treated with LFA+hTERT (first bar) or LFA+hTERT+B18R (second bar).

FIG. 5 is a bar graph showing IL-8 secretion (pg/1000 cells) from chondrocytes 18 hours after transfection with lipofectamine (first bar), nGFP (second bar), hTERT CI (third bar), or hTERT WT (fourth bar) with or without B18R and p65i treatment.

FIG. 6 is a bar graph showing chondrocyte doubling times at passage 7 after treatment with control (first bar), hTERT CI (second bar), or hTERT WT (third bar) at passage 5 (first set) or passages 5 and 6 (second set).

FIG. 7 is a bar graph showing RANTES levels (pg/5000 cells) after treatment with control (first bar), lipofectamine (second bar), hTERT mmRNA (third bar), Tofacitinib (fourth bar), or Curcurbitacin (fifth bar) 0 hours (first set) and 18 hours (second set) after treatment.

FIG. 8 is a bar graph showing RANTES levels (pg/5000 cells) after treatment with control (first bar), lipofectamine (second bar), hTERT mmRNA (third bar), Aspirin (fourth bar), or Celecoxib (fifth bar), or IL-10 (sixth bar) 0 hours (first set) and 18 hours (second set) after treatment.

### DETAILED DESCRIPTION

Telomeres are DNA sequences at the ends of chromosomes that protect the ends of the chromosomes but that shorten over time. Critically short telomeres may cause cells to stop functioning correctly or to die. Critically short telomeres may also lead to chromosome fusions that may in turn lead to cancer. Even in the absence of a specific diagnosed disease, short telomeres are implicated in the gradual decline in function of the mind and body and in the appearance of aging.

In mammals, telomeres comprise tandem repeats of the sequence TTAGGG, and in other animals such as birds, reptiles, and fish, the repeated sequence varies. In all of these types of animals, the telomeres are double stranded for many kilobases (kb). Average telomere lengths differ between species, and among individuals of a species. In humans, telomeres start out before birth with lengths of 15-20 kb, and at birth with lengths of 12-15 kb. Telomeres shorten rapidly during childhood, and then by about 0-100 bp per year on average in adulthood, a rate which varies depending on the cell type, exposure to psychological or oxidative stress, and other factors.

Telomeres are part of the telomere complex, which protects the ends of chromosomes. The telomere complex also comprises a set of proteins collectively called Shelterin. Telomere complex proteins include POT1, TPP1, ATM, DAT, 10 TRF1, TRF2, Rap1, Rif1, TIN2, NBS, MRE17, and RAD50 and their homologs in different mammalian species. In many species the telomere terminates in a single-stranded 3′ overhang which inserts itself into the double stranded region, in association with telomere complex proteins, forming a loop within the telomere complex.

Telomeres shorten over time, due to oxidative damage and sister chromatid exchange, and also due to the end replication problem, in which the ends of chromosomes are not completely duplicated during mitosis. When telomeres become critically short, the telomere complex is no longer able to protect the chromosome ends, and the chromosome ends become “uncapped”. Uncapping of the chromosome ends may result in chromosome-chromosome fusions, which may in turn result in cancer. Uncapping can also result in the chromosome ends being recognized as damaged DNA, activating DNA damage responses and triggering cell apoptosis or senescence. Senescence is an arrested state in which the cell remains viable but no longer divides, and senescent cells typically cease to perform their normal, pre-senescence, useful functions adequately or at all. Thus, telomere shortening leads to tissue dysfunction, loss of physical ability and youthful appearance, loss of mental ability, and disease in part due to the accumulation of senescent cells, and in part due to the loss of cells by apoptosis. Indeed, aged people with short telomeres are approximately 200-750% more likely to develop myocardial infarction (200%), vascular dementia (200%), diabetes with complications (400%), cancer, stroke, Alzheimer's disease, infection (750%), idiopathic pulmonary fibrosis, and other disease. People with short telomeres in one tissue are likely to also have short telomeres in most of their other tissues, and thus short telomeres correlate with increased risk for many diseases in one individual. Intriguingly, there are areas of focal senescence in humans. For example, at bends or bifurcations of blood vessels, the endothelial cells lining the vessel show signs of accelerated aging with shorter telomeres. Short telomeres also limit cell replicative capacity which in turn limits cell therapies and regenerative therapies. Conversely, in mice with genetically-induced short telomeres, increasing their telomere length using virus-based genetic engineering methods rejuvenates the mice by several parameters, including skin thickness and elasticity, liver function, and sense of smell.

Since telomerase extends telomeres, a useful approach to extending telomeres is to increase the level of telomerase activity in cells. Many agents and conditions have been reported to increase telomerase activity in cells, including the agents and conditions listed in Table 1.

TABLE 1
Examples of agents and conditions that increase telomerase activity

| Type | Examples |
|---|---|
| Growth factors | EGF, IGF-1, FGF-2, VEGF |
| Genetic | Viral delivery of DNA encoding TERT, |
| treatments | electroporation of plasmid encoding TERT, |
| | transfection of mRNA encoding TERT |
| Hormones | Estrogen, erythropoietin |
| Physical | UV radiation, hypoxia |
| treatments | |
| Cytokines | IL-2, IL-4, IL-6, IL-7, IL-13, and IL-15 |
| Small molecules | Resveratrol, compounds extracted from |
| from plants | Astragalus membranaceus including |
| | cycloastragenol (TAT2), TA-65, or TAT153 |
| Other | Inhibitors of Menin, SIP1, pRB, p38, p53, p73, |
| | MKRN1, CHIP, Hsp70, androgens, and TGF-beta |

The treatment examples of Table 1 are not without undesired effects, however. For example, treatment with growth factors, hormones, or cytokines may cause side effects, may activate multiple signaling pathways, may cause unwanted cell replication, may trigger an immune response, and are generally non-specific. Genetic treatments using plasmids or viruses carry a risk of genomic modification by insertional mutagenesis and a risk of cancer. Transfection with unmodified RNA causes a strong immune response and has not been shown to extend telomeres. Physical treatments can damage genomic DNA. Treatment with small molecules from plants have been found to only extend telomeres in some subjects and cells, only extend telomeres very slowly, and require chronic delivery, therefore risking cancer.

The expression in cells of nucleic acid sequences encoding hTERT and TERC, and the use of these components themselves, have been proposed to be useful in the diagnosis, prognosis, and treatment of human diseases (see, e.g., U.S. Pat. Nos. 5,583,016 and 6,166,178), but telomere extension in a manner that is both rapid and transient, and thus potentially safe for the reasons described above, has not been demonstrated. Sæbe-Larssen et al. (2002) J. Immunol. Methods 259:191-203 reported the transfection of dendritic cells with mRNA encoding hTERT, and that such cells acquired telomerase activity, but the transfection used standard mRNA and resulted in a strong hTERT cytotoxic T lymphocyte (CTL) response rather than an extension of telomeres.

Furthermore, all existing small-molecule treatments are largely ineffective and slow, primarily because they act through the catalytic component of telomerase, TERT, which is heavily regulated post-translationally, limiting existing treatments' effects to a small subset of cells, and excluding cells in interphase or G0 such as many stem and progenitor cells. This regulation is mediated in part by interactions between components of the telomerase complex, the telomere complex, and other molecules. For example, TERT is phosphorylated or dephosphorylated at multiple sites by multiple kinases and phosphatases, and at some sites, phosphorylation results in increased telomerase activity (for example phosphorylation by Akt), while at others sites phosphorylation reduces telomerase activity (for example, phosphorylation by Src1 or cAb1). Also, TERT is ubiquitinated or deubiquitinated at specific sites. TERT also interacts with other proteins at specific sites on TERT, and these interactions can inactivate TERT (for example interactions with Pinx1 or cAb1), or transport TERT away from the chromosomes (for example, interactions with CRM1 and Pinx1), preventing or lowering telomere extension. Further, some proteins bind to telomeres or the telomere complex, blocking TERT (for example POT1), preventing telomere extension. Further, some proteins aid telomere extension indirectly, for example helicases and UPF1. Due to regulatory mechanisms, telomerase activity peaks during S phase of the cell cycle, and thus rapidly-dividing cells may tend to benefit more from treatments that increase telomerase activity. However, it is often desirable to keep cells in a slow-dividing or non-dividing state; for example, stem or progenitor cells are often slow-dividing, and thus may spend the majority of their time in interphase or G0. Thus, existing treatments are slow and ineffective in most cell types generally, and in all cell types during interphase and G0. Treatments that are slow are less safe, because they require treatment for a longer time. Since telomere-shortening provides a protective safety mechanism against run-away cell proliferation, such as in cancer, a treatment that extends telomeres rapidly is generally safer, because it may be delivered for short periods of time and infrequently, thus allowing the normal telomere-shortening safety mechanism to remain in effect for much of the time. Therefore a method capable of transiently overcoming telomerase regulation to rapidly extend telomeres during a brief treatment is needed.

TERT regulates hundreds of genes including those listed in Table 2.

TABLE 2
Examples of genes and pathways regulated by TERT

| | |
|---|---|
| Type | Examples |

Upregulated

| | |
|---|---|
| Epigenetic state modulators | DNA 5-methylcytosine transferase I |
| Proto-oncogenes | Hepatocyte growth factor receptor (MET), |
| | AKT-2, CRK |
| Differentiation, cell fate | Sox-13, Wnt |
| Glycolysis | Phosphofructokinase, aldolase C |
| Proliferation enhancers | Activating transcription factor-3, Xbox |
| | protein-1, FGF, EGFR, Insulin-like growth |
| | factor 2, Wnt, tp53bp1, epiregulin |
| Metastasis-related genes | Mac-2 binding protein |

Downregulated

| | |
|---|---|
| Proliferation inhibitors | Interleukin 1 receptor antagonist, |
| | parathyroid hormone-related peptide, |
| | integrin-associated protein, TNF-related |
| | apoptosis-inducing ligand, IGF binding |
| | protein-5, Melanoma inhibitory activity, |
| | p21, p53 |
| Differentiation, cell fate | Transforming growth factor B2 |

In many cases, modulating the genes or pathways of Table 2 is undesirable because doing so can cause unwanted changes in cells. For example, TERT activates epigenetic regulators, which can change cell phenotype or interfere with efforts to reprogram or transdifferentiate cells for therapeutic purposes. TERT activates growth enhancers, but often proliferation is not desired, for example often stem cells with the most regenerative potential are those which divide slowly. TERT modulates regulators of cell fate and differentiation, which can impair efforts to differentiate cells into specific cell types. TERT also activates proto-oncogenes, which could lead to cancer. Thus, it is desirable to minimize the amount of time during which TERT levels are artificially elevated, including any treatment that extends telomeres using TERT. A treatment that extends telomeres by only transiently increasing telomerase activity levels is therefore needed.

In some cell types TERT has been shown to affect expression of other genes, and this may not be desirable in some cases. Thus, a treatment that minimizes the amount of time during which TERT levels are increased is needed.

Compositions

Compositions are disclosed for the transient expression of exogenous telomerase in an RNA immune-responsive cell, such as a chondrocyte. The term “RNA immune-responsive cell” refers to a cell where there is an innate immunity response to synthetic messenger RNA.

In some cases, the RNA immune-responsive cell is an mmRNA immune-responsive cell. The term “mmRNA immune-responsive cell” refers to a cell where there is an innate immunity response to modified messenger RNA.

The compounds comprise a synthetic ribonucleic acid comprising at least one synthetic RNA encoding a telomerase reverse transcriptase (TERT RNA), and an anti-inflammatory agent, wherein telomeres are extended within an RNA immune-responsive cell treated with the composition.

Synthetic Ribonucleic Acids

The ribonucleic acids used in the transient expression of TERT can comprise a synthetic ribonucleic acid encoding a TERT protein. The ribonucleic acids typically further comprise sequences that affect the expression and/or stability of the ribonucleic acid in the cell. For example, the ribonucleic acids may contain a 5′ cap and untranslated region (UTR) to the 5′ and/or 3′ side of the coding sequence. The ribonucleic acids may further contain a 3′ tail, such as a poly-A tail. The poly-A tail may, for example, increase the stability of the ribonucleic acid. In some embodiments, the poly-A tail is at least 75 nucleotides, 100 nucleotides, 125 nucleotides, 150 nucleotides, or even longer.

In some embodiments, the 5′ cap of the ribonucleic acid is a non-immunogenic cap. In some embodiments, the 5′ cap may increase the translation of the ribonucleic acid. In some embodiments, the 5′ cap may be treated with phosphatase to modulate the innate immunogenicity of the ribonucleic acid. In some embodiments, the 5′ cap is an anti-reverse cap analog (“5 ARCA”), such as a 3′-O-Me-m7G(5′)ppp(5′)G RNA cap structure analog.

As is well-known in the art, the above features, or others, may increase translation of the TERT protein encoded by the ribonucleic acid, may improve the stability of the ribonucleic acid itself, or may do both. In some embodiments, the 5′ UTR and/or the 3′ UTR are from a gene that has a very stable mRNA and/or an mRNA that is rapidly translated, for example, a-globin or β-globin, c-fos, or tobacco etch virus. In some embodiments, the 5′ UTR and 3′ UTR are from different genes, or are from different species than the species into which the compositions are being delivered. The UTRs may also be assemblies of parts of UTRs from the mRNAs of different genes, where the parts are selected to achieve a certain combination of stability and efficiency of translation.

The ribonucleic acids may be nucleoside-modified RNA (“modRNA”). Most mature RNA molecules in eukaryotic cells contain nucleosides that are modified versions of the canonical unmodified RNA nucleosides, adenine, cytidine, guanosine, and uridine. Those modifications may prevent the RNA from being recognized as a foreign RNA. Synthetic RNA molecules made using certain nucleosides are much less immunogenic than unmodified RNA. The immunogenicity can be reduced even further by purifying the synthetic modRNA, for example by using high performance liquid chromatography (HPLC). The modified nucleosides may be, for example, chosen from the nucleosides shown in Table 3. The nucleosides are, in some embodiments, pseudouridine, 2-thiouridine, or 5-methylcytidine. Under some circumstances, it may be desirable for the modified RNA to retain some immunogenicity.

TABLE 3
Modified nucleosides found in eukaryotic RNA

| symbol | common name |
|---|---|
| m¹A | 1-methyladenosine |
| m⁶A | N⁶-methyladenosine |
| Am | 2′-O-methyladenosine |
| i⁶A | N⁶-isopentenyladenosine |
| io⁶A | N⁶-(cis-hydroxyisopentenyl)adenosine |
| ms²io⁶A | 2-methylthio-N⁶-(cis-hydroxyisopentenyl) adenosine |
| g⁶A | N⁶-glycinylcarbamoyladenosine |
| t⁶A | N⁶-threonylcarbamoyladenosine |
| ms²t⁶A | 2-methylthio-N⁶-threonyl carbamoyladenosine |
| Ar(p) | 2′-O-ribosyladenosine (phosphate) |
| m⁶₂A | N⁶,N⁶-dimethyladenosine |
| m⁶Am | N⁶,2′-O-dimethyladenosine |
| m⁶₂Am | N⁶,N⁶,2′-O-trimethyladenosine |
| m¹Am | 1,2′-O-dimethyladenosine |
| m³C | 3-methylcytidine |
| m⁵C | 5-methylcytidine |
| Cm | 2′-O-methylcytidine |
| ac⁴C | N⁴-acetylcytidine |
| f⁵C | 5-formylcytidine |
| m⁴C | N⁴-methylcytidine |
| hm⁵C | 5-hydroxymethylcytidine |
| f⁵Cm | 5-formyl-2′-O-methylcytidine |
| m¹G | 1-methylguanosine |
| m²G | N²-methylguanosine |
| m⁷G | 7-methylguanosine |
| Gm | 2′-O-methylguanosine |
| m²₂G | N²,N²-dimethylguanosine |
| Gr(p) | 2′-O-ribosylguanosine (phosphate) |
| yW | wybutosine |
| o₂yW | peroxywybutosine |
| OHyW | hydroxywybutosine |
| OHyW* | undermodified hydroxywybutosine |
| imG | wyosine |
| m^{2,7}G | N²,7-dimethylguanosine |
| m^{2,2,7}G | N²,N²,7-dimethylguanosine |
| I | inosine |
| m¹I | 1-methylinosine |
| Im | 2′-O-methylinosine |
| Q | queuosine |
| galQ | galactosyl-queuosine |
| manQ | mannosyl-queuosine |
| Ψ | pseudouridine |
| D | dihydrouridine |
| m⁵U | 5-methyluridine |
| Um | 2′-O-methyluridine |
| m⁵Um | 5,2′-O-dimethyluridine |
| m¹Ψ | 1 -methylpseudouridine |
| Ψm | 2′-O-methylpseudouridine |
| s²U | 2-thiouridine |
| ho⁵U | 5-hydroxyuridine |
| chm⁵U | 5-(carboxyhydroxymethyl)uridine |
| mchm⁵U | 5-(carboxyhydroxymethyl)uridine methyl ester |
| mcm⁵U | 5-methoxycarbonylmethyluridine |
| mcm⁵Um | 5-methoxycarbonylmethyl-2′-O-methyluridine |
| mcm⁵s²U | 5-methoxycarbonylmethyl-2-thiouridine |
| ncm⁵U | 5-carbamoylmethyluridine |
| ncm⁵Um | 5-carbamoylmethyl-2′-O-methyluridine |
| cmnm⁵U | 5 -carboxymethylaminomethyluridine |
| m³U | 3-methyluridine |
| m¹acp³Ψ | 1-methyl-3-(3-amino-3-carboxypropyl) pseudouridine |
| cm⁵U | 5-carboxymethyluridine |
| m³Um | 3,2′-O-dimethyluridine |
| m⁵D | 5-methyldihydrouridine |
| τm⁵U | 5-taurinomethyluridine |
| τm⁵s²U | 5-taurinomethyl-2-thiouridine |

Without intending to be bound by theory, the presence of the modified nucleosides enables modRNA to avoid activation of an immune response mediated by various receptors, including the Toll-like receptors and RIG-1. Nonimmunogenic modRNA has been used as a therapeutic agent in mice via topical delivery. The discovery of nucleotide-modified mRNA facilitates the delivery of RNA-encoded therapeutic proteins, or mutants thereof, to cells, and the expression of those proteins in cells.

Accordingly, in some embodiments, the ribonucleic acids of the instant compositions comprise a pseudouridine, a 2-thiouridine, a 5-methylcytidine, or a nucleoside from Table 3. In some embodiments, the ribonucleic acids comprise more than one of the above nucleosides or combination of the above nucleosides. In some embodiments, the ribonucleic acids comprise pseudouridine and 5-methylcytidine.

In some embodiments, an immune response to the modRNA may be desired, and the RNA may be modified to induce an optimal level of innate immunity. In other embodiments, an immune response to the modRNA may not be desired, and the RNA may be modified in order to minimize such a reaction. The RNA can be modified for either situation.

The ribonucleic acids of the instant compositions may be synthetic ribonucleic acids. The term “synthetic”, as used herein, means that the ribonucleic acids are in some embodiments prepared using the tools of molecular biology under the direction of a human, for example as described below. The synthetic ribonucleic acids may, for example, be prepared by in vitro synthesis using cellular extracts or purified enzymes and nucleic acid templates. The synthetic ribonucleic acids may in some embodiments be prepared by chemical synthesis, either partially or completely. Alternatively, or in addition, the synthetic ribonucleic acids may in some embodiments be prepared by engineered expression in a cell, followed by disruption of the cell and at least partial purification of the ribonucleic acid. A synthetic ribonucleic acid is not, however, a naturally-occurring ribonucleic acid, as it is expressed in an unmodified cell without extraction or purification.

The ribonucleic acids of the instant invention may be prepared using a variety of standard techniques, as would be understood by one of ordinary skill in the art. In some embodiments, the ribonucleic acids may be prepared by in vitro synthesis, as described, for example, in U.S. Pat. Nos. 8,278,036 and 9,012,219. In some embodiments, the ribonucleic acids may be prepared by chemical synthesis. In some embodiments, the ribonucleic acids may be prepared by a combination of in vitro synthesis and chemical synthesis. As described above, the term “synthetic” should be understood to include ribonucleic acids that are prepared either by chemical synthesis, by in vitro synthesis, by expression in vivo and at least partial purification, or by a combination of such, or other, chemical or molecular biological methods.

The ribonucleic acids of the instant invention may, in some embodiments, be purified. As noted above, purification may reduce immunogenicity of the ribonucleic acids and may be advantageous in some circumstances. See also U.S. Pat. No. 9,012,219. In preferred embodiments, the ribonucleic acids are purified by HPLC or by affinity capture and elution.

The protein structure of TERT includes at least three distinct domains: a long extension at the amino-terminus (the N-terminal extension, NTE) that contains conserved domains and an unstructured linker region; a catalytic reverse-transcriptase domain in the middle of the primary sequence that includes seven conserved RT motifs; and a short extension at the carboxyl-terminus (the C-terminal extension, CTE). In some embodiments, the ribonucleic acid of the instant invention codes for a full-length TERT. In some embodiments, the ribonucleic acid codes for a catalytic reverse transcriptase domain of TERT. In some embodiments, the ribonucleic acid codes for a polypeptide having TERT activity.

The TERT encoded by the ribonucleic acids of the disclosed compositions may be a mammalian, avian, reptilian, or fish TERT; or chimeric form containing sequences from different species; or another variant that retains telomerase activity. For example, the TERT can be a human TERT. The amino acid sequence of two human TERT isoforms are available as NCBI Reference Sequences: NP_937983.2 and NP_001180305.1. Other non-limiting exemplary amino acid sequences usefully encoded by the ribonucleic acids of the instant compositions include TERT from cat (NCBI Reference Sequence: XP_003981636.1), dog (NCBI Reference Sequence: NP_001026800.1), mouse (NCBI Reference Sequence: NP_033380.1), cow (NCBI Reference Sequence: NP_001039707.1), sheep NCBI Reference Sequence: XP_004017220.1), pig (NCBI Reference Sequence: NP_001231229.1), African elephant (NCBI Reference Sequence: XP_003408191.1), chicken (NCBI Reference Sequence: NP_001026178.1), rat (NCBI Reference Sequence: NP_445875.1), zebrafish (NCBI Reference Sequence: NP_001077335.1); Japanese medaka (NCBI Reference Sequence: NP_001098286.1); and chimpanzee (NCBI Reference Sequences: XP_003950543.1 and XP_003950544.1).

It should be understood that the disclosed ribonucleic acids may code for variants of any of the above-listed amino acid sequences, particularly variants that retain telomerase catalytic activity, including truncated variants. In some embodiments, the ribonucleic acids of the instant compositions code for one of the above-listed amino acid sequences or a sequence with at least 95% sequence identity to that sequence. In some embodiments, the nucleic acids of the instant compositions code for one of the above-listed amino acid sequences or a sequence with at least 98%, 99%, 99.9%, or even higher sequence identity to that sequence.

It should also be understood that the instant ribonucleic acids may correspond to the native gene sequences coding for the above-listed TERT proteins or may correspond to variants that are made possible due to the redundancy of the genetic code, as would be understood by one of ordinary skill in the art. In some embodiments, the codon selection may be optimized to optimize protein expression using algorithms and methods known by those of ordinary skill in the art.

In some embodiments, the disclosed compositions further comprise a telomerase RNA component (TERC).

Anti-Inflammatory Agent

In some embodiments, the anti-inflammatory agent comprises an interferon antagonist, such as the B18R protein. In some embodiments, the anti-inflammatory agent comprises an NFκB antagonist, such as the RelA/NFkB p65 [p Ser529, p Ser536] inhibitor peptide.

In some embodiments, the anti-inflammatory agent comprises a Jak-Stat inhibitor, such as Tofacitinib, Curcurbitacin, or a combination thereof. In some embodiments, the anti-inflammatory agent comprises a non-steroidal anti-inflammatory drug (NSAID), such as Indomethacin, Ibuprofen, or Celecoxib. In some embodiments, the anti-inflammatory agent comprises a steroid, such as celastrol, dexamethasone, or prednisone. In some embodiments, the anti-inflammatory agent comprises an immunosuppressant, such as rapamycin, everolimus, or a combination thereof. In some embodiments, the anti-inflammatory agent comprises an anti-inflammatory cytokine, such as IL-10. In some embodiments, the anti-inflammatory agent comprises an analgesic, such as aspirin, acetaminophen, or a combination thereof. In some embodiments, the anti-inflammatory agent may be a naturally occurring anti-inflammatory, such as fish oil (omega 3 fatty acid).

Delivery Vehicles

In some embodiments, the compositions further comprise a delivery vehicle for the ribonucleic acid. The delivery vehicle may, in some cases, facilitate targeting and uptake of the ribonucleic acid of the composition to the target cell. In particular, the compositions of the instant disclosure may comprise any gene delivery vehicle known in the field, for example nanoparticles, liposomes, gene gun ballistic particles, cell squeezer, nucleofection, viruses, cationic lipids, commercial products, such as Lipofectamine® RNAiMax, or other vehicles. In some embodiments, the delivery vehicle is an exosome, a lipid nanoparticle, a polymeric nanoparticle, a natural or artificial lipoprotein particle, a cationic lipid, a protein, a protein-nucleic acid complex, a liposome, a virosome, or a polymer. In some embodiments, the delivery vehicle is a cationic lipid formulation. In some cases, the delivery vehicle comprises a liposome that comprises DOTAP and cholesterol in a 1:1 ratio. In some cases, a positively charged protein, such as protamine, that can facilitate the loading and binding of mmRNA to the liposome.

In some embodiments, the delivery vehicle is an exosome, a lipid nanoparticle, or a polymeric nanoparticle. Exosomes are naturally-occurring lipid bilayer vesicles 40-100 nm in diameter. Exosomes contain a set of specific proteins, including the membrane protein Lamp-1 and Lamp-2, which are particularly abundant. In 2007, exosomes were discovered to be natural carriers of RNA and protein, including over 1,300 types of mRNA and 121 types of non-coding microRNA. Exosomes can also transmit mRNA between species: exposure of human cells to mouse exosomes carrying mouse mRNA results in translation in the human cells of the mouse mRNA.

As delivery vehicles for RNA, protein, or DNA, exosomes have a number of advantages over alternative vehicles. Specifically, exosomes can be generated from a patient's own cells, making them non-immunogenic—they are therefore not attacked by antibodies, complement, coagulation factors, or opsonins. In addition, exosomes can be loaded with nucleic acids by electroporation, and they are naturally-occurring vehicles that carry mRNA and protein between human cells. Exosomes protect their RNA and protein cargo during transport, and the cargo is delivered directly into the cytosol. They can extravasate from the blood stream to extravascular tissues, even crossing the blood-brain barrier, and they can be targeted. Furthermore, exosomes avoid being accumulated in untargeted organs, such as, for example, liver. Exosomes may therefore be used as cell-derived “liposomes” to deliver therapeutic mRNA or other cargo in the treatment of disease.

Most cell types are believed to be capable of generating exosomes, and exosomes are found in most biological fluids including blood, saliva, urine, cerebrospinal fluid, breast milk, and amniotic fluid. Exosomes are produced by most cell types, in different abundance. Abundant exosomes, devoid of T-cell activators, can be derived from immature dendritic cells, which are present in human blood. Exosomes may also be produced artificially, for example by combining recombinant exosomal proteins with lipids and phospholipids such as are found in exosomal membranes. Alternatively, exosomes may be constructed by in vitro self-assembly of liposomes with a subset of exosomal surface proteins.

The drug delivery potential of exosomes was first demonstrated in 2011. Specifically, exosomes were harvested from dendritic cells engineered to express a Lamp2B fusion protein fused to a 28 a.a. targeting ligand from rabies virus glycoprotein (RVG). siRNA was then electroporated into the exosomes and the exosomes injected into mice immunocompatible with the mice from which they obtained the dendritic cells. The exosomes were thus autologous, and did not generate an immune response, as measured by IL-6, IP-10, TNF-α, and IFN-α levels. Further, repeated doses over one month elicited similar responses, demonstrating that there was no adaptive immune response either.

As described above, exosomes can be autologous and thus have low immunogenicity. Since modRNA also has low immunogenicity, the combination of modRNA as the ribonucleic acid and an exosome as the delivery vehicle in the compositions of the instant disclosure is particularly preferred. In these embodiments, the disclosure thus provides a new way of delivering mRNA or modRNA to cells or tissues, using exosomes. Such delivery provides a useful method to temporarily increase the level of any protein in a cell in vivo using RNA delivered in exosomes by intravenous or topical injection, and particularly in the delivery of an RNA encoding TERT. Accordingly, in preferred embodiments, the delivery vehicles of the instant compositions are non-immunogenic. Under some circumstances, however, it may be desirable for the vehicle to retain some immunogenicity.

Additional Components

The compositions disclosed herein may further comprise additional components that either enhance the delivery of the composition to the target cell, enhance the extension of telomeres within the cell, or both. For example, the compositions may further comprise one or more of the compounds and conditions of Table 1. As would be understood by one of ordinary skill in the art, combinations of active ingredients often display synergistic effects on a desired activity, such as, for example, the transient expression of exogenous telomerase activity in a cell, and such combinations are understood to fall within the scope of the invention. Additional examples of proteins that may be included within the compositions of the instant disclosure are listed in Table 4. It should be understood that the compositions could either include the proteins themselves, or nucleic acid sequences, such as RNAs or modRNAs, that encode these proteins, or proteins with high sequence identity that retain the activity of the listed protein.

TABLE 4
Proteins usefully delivered in combination with TERT

| Protein | Activity | Advantages |
|---|---|---|
| UPF1 | Sustains telomere leading strand-replication | Increased rate or amount |
| | | of telomere extension. |
| HSP90 | Prevents dephosphorylation of Akt kinase by | Increased TERT |
| | PP2A. Akt needs to be phosphorylated to | activity. |
| | phosphorylate TERT. Also complexes with TERT | |
| | and keeps TERT serine 823 phosphorylated, | |
| | keeping TERT activated. | |
| Akt kinase | Complexes with TERT and HSP90, | Increased TERT |
| (aka protein | phosphorylates TERT at serine 823, increasing | activity. |
| kinase B) | TERT activity. | |
| Protein kinase C | Phosphorylates TERT, allowing it to bind nuclear | Nuclear translocation. |
| (PKC) | translocator. | |
| (its various | | |
| isoenzymes) | | |
| Shp-2 | Inhibits phosphorylation of TERT Y707 by Src1, | Nuclear translocation. |
| | keeping TERT in nucleus. Transport of TERT to | |
| | nucleus. | |
| TPP1 | Recruits telomerase to the telomere. | |
| NFkB p65 | Transport of TERT to nucleus. | Nuclear translocation. |
| Rap1 | regulator of telomere length. | Extension of telomeres. |

Other examples of agents that may usefully be included within the compositions of the instant disclosure are listed in Table 5.

TABLE 5
Other agents usefully delivered in combination with TERT

| Molecule | Activity | Advantages |
|---|---|---|
| Okadaic acid | Inhibits PP2A. PP2A | Increased telomerase activity due to |
| | dephosphorylates AKT and or | TERT phosphorylation. |
| | TERT. AKT phosphorylates TERT, | |
| | activating it. | |
| TERRA or anti- | TERRA inhibits telomerase by | Antisense TERRA, or ARRET, |
| sense TERRA | binding to TERC, to which it is | should increase telomerase activity by |
| (ARRET) | complementary. | binding to TERRA, preventing it from |
| | | binding to TERC. |
| TERC | RNA component of telomerase, | Increase telomerase activity. |
| RNA | essential for its function, may be | |
| | second-most limiting factor after | |
| | TFRT in most cells. | |

Since TERT is most active during certain phases of the cell cycle, the compositions of the instant disclosure may also optionally include one or more transient activators of cellular proliferation, in order to enhance the effectiveness of the TERT treatment. Such agents may include, for example, an RNAi agent that transiently reduces the amounts of cell cycle inhibitors such as Rb or P19/Arf in the cell. Other transient activators of cellular proliferation may be usefully included in the instant compositions, as would be understood by one of ordinary skill in the art.

Methods of Extending Telomeres and Methods of Treatment

In another aspect, the instant disclosure provides methods of extending telomeres, comprising the step of administering any of the above-described compounds or compositions to a RNA immune-responsive cell with shortened telomeres, wherein telomeres are extended within the RNA immune-responsive cell. The instant disclosure also provides methods of treatment, comprising the step of administering any of the above-described compounds or compositions to an animal subject in need of, or that may benefit from, telomere extension.

In some embodiments, the compounds or compositions are administered to a RNA immune-responsive cell, wherein the RNA immune-responsive cell is an isolated cell or is part of a cell culture, an isolated tissue culture, or an isolated organ (i.e., administration is in vitro). In some embodiments, the compounds or compositions are administered without isolating the cell or cells, the tissue, or the organ from the subject (i.e., the administration is in vivo). In some of these embodiments, the compound or composition is delivered to all, or almost all, RNA immune-responsive cells in the subject's body. In some embodiments, the compound or composition is delivered to a specific cell or tissue in the subject's body.

In some embodiments, the subject is a mammal, bird, fish, or reptile. In some embodiments, the subject is a human. In some embodiments, the subject is a pet animal, a zoo animal, a livestock animal, or an endangered species animal.

For in vitro applications, the compounds or compositions may be administered using any suitable technique, as would be understood by those skilled in the fields of cell biology, cell culture, tissue culture, organ culture, or the like. For in vivo applications, the compounds or compositions are usefully administered by injection, topical application, inhalation, or any other suitable administration technique, as would be understood by those of ordinary skill in the medical arts or the like.

As described above, cells usefully treated according to the methods of the disclosure include cells, either in a subject (for in vivo administration) or from a subject (for in vitro administration), that may benefit from either as a preventive measure, for example to prevent or delay onset of the administration is in vitro).

In other embodiments, the compounds or compositions are administered without isolating the RNA immune-responsive cell or cells. In this case it is the cells within the tissue (s), the organ(s), or the whole organism that are treated. Since short telomeres affect almost all cell types in most mammals, telomere extension may benefit most mammals. A telomere extension treat many diseases and conditions in which short telomeres are implicated, or as a treatment for those diseases and conditions. The treatment may benefit subjects at risk of age-related diseases or conditions involving RNA immune-responsive cells, such as chondrocytes, or who are already suffering from such diseases, and may also benefit subjects who have experienced, are experiencing, or are at risk of experiencing physical trauma or chronic physical stress such as hard exercise or manual labor, or psychological trauma (such as post-traumatic stress disease) or chronic psychological stress (such as childhood abuse or neglect), since all of these conditions cause telomere shortening; physical stress or trauma requires cell division in order to repair the resultant damage, thus shortening telomeres, and these conditions may also cause oxidative stress, which also shortens telomeres.

In some embodiments, the RNA immune-responsive cell treated according to the instant methods are from subjects where no disease state is yet manifested but where the subject is at risk for a condition or disease involving short telomeres, or where the cells contain shortened telomeres. In some embodiments, the age-related illness is simply old age. In cases of physical trauma such as a bone fracture or a tissue crush or cut injury or burn, the invention may be used to increase the lengths of telomeres in RNA immune-responsive cell which participate in healing the trauma, to increase their replicative capacity. In cases of chronic physical stress, which causes telomere shortening, treatment with the invention may lengthen telomeres in affected RNA immune-responsive cell increasing their replicative capacity and ability to repair tissue damage. The treatment methods may also be useful in advance of or during surgery or chemotherapy, or radiotherapy, to increase the ability of RNA immune-responsive cells to replicate to repair damage resulting from these procedures.

The methods may also be useful for treating RNA immune-responsive cells in vitro for various applications, including autologous or heterologous cell therapy, bioengineering, tissue engineering, and growth of artificial organs, tissues or limbs. In these applications, cells may be required to divide many times, which may lead to loss of telomere length, which may be counteracted by the disclosed compositions and methods before, during, or after the application.

Administration

The administering step may be performed one or more times depending on the amount of telomere extension desired. In some embodiments of the instant methods, the RNA immune-responsive cell is an isolated cell, and the administering step lasts no longer than 96 hours, no longer than 72 hours, no longer than 48 hours, no longer than 36 hours, no longer than 24 hours, no longer than 18 hours, no longer than 12 hours, no longer than 8 hours, no longer than 4 hours, or even shorter times. In some embodiments, the administering step lasts at least 2 hours, at least 4 hours, at least 8 hours, at least 12 hours, at least 18 hours, at least 24 hours, at least 36 hours, at least 48 hours, or even longer times. In preferred embodiments, the administering step lasts no longer than 48 hours, no longer than 96 hours, or no longer than 1 week. In other preferred embodiments, the administering step lasts at least 2 hours. It should be understood that, in the case where administration is by transfection, the time for administration includes the time for the cell to recover 20 from the transfection method.

In some embodiments of the instant methods, the RNA immune-responsive cell is an isolated RNA immune-responsive cell, and the administering step is performed no more than 6 times, no more than 5 times, no more than 4 times, no more than 3 times, no more than 2 times, or even no more than 1 time. In some embodiments, the administering step is performed not less than 2 times, not less than 3 times, not less than 4 times, not less than 5 times, not less than 6 times, or even more often.

In some embodiments, the administering step is performed once or a few times over a relatively brief period to re-extend telomeres, and then not performed for a prolonged period until telomeres need to be extended again. This cycle may be repeated indefinitely. Such a treatment schedule allows telomeres to be periodically re-extended, with intervals in between administration steps during which telomeres shorten. Periodic treatment methods may be performed either by in vivo administration or by in vitro administration, as desired. In some embodiments, the administering step in such a series is performed no more than 6 times, no more than 5 times, no more than 4 times, no more than 3 times, no more than 2 times, or even no more than 1 time. In some embodiments, the administering step is performed not less than 2 times, not less than 3 times, not less than 4 times, not less than 5 times, not less than 6 times, or even more often. By varying the number of times the administering step is performed, and the dose of the disclosed compounds or compositions used, the amount of telomere extension achieved can be controlled.

In some embodiments, the disclosed methods further include the step of culturing the RNA immune-responsive cell on a specific substrate, preferably an elastic substrate. Such substrates are known to prevent unwanted changes in the RNA immune-responsive cell that would normally occur on other substrates due to the non-physiological elasticity of those substrates. See US Patent Publication No. 2012/0177611, which is incorporated by reference herein in its entirety. Elastic substrates may additionally promote cell survival.

Administration of the compounds or compositions of the instant disclosure results in the transient expression of a telomerase activity in the RNA immune-responsive cell. The increased activity is readily measured by various assays, such as, for example, the Trapeze® 20 RT telomerase detection kit (Millipore), which provides a sensitive, real-time in vitro assay using fluorimetric detection and quantitation of telomerase activity, although other measurement techniques are also possible. In some embodiments, the telomerase activity is increased by at least 5%, at least 10%, at least 20%, at least 30%, at least 50%, or even more. In preferred embodiments, the telomerase activity is increased by at least 5%.

As previously noted, one of the advantages of the instant techniques is that the expression of telomerase activity is transient in the treated cells. In particular, such transient expression is in contrast to previous techniques where a telomerase reverse transcriptase gene is inserted into the genomic sequence of the cell or otherwise permanently modifies the genetic make-up of the targeted cell and results in constitutive activity of the nucleic acid sequence.

The speed of telomere extension made possible with the disclosed compounds, compositions, and methods enables telomere maintenance by very infrequent delivery of TERT modRNA. The expressed telomerase activity rapidly extends telomeres in a brief period, before being turned over, thus allowing the protective mechanism of telomere-shortening to function most of the time. Between treatments, normal telomerase activity and telomere shortening is present, and therefore the anticancer safety mechanism of telomere shortening to prevent out-of-control proliferation remains intact, while the risk of short telomere-related disease remains low. In contrast, the best existing small molecule treatment for extending telomeres requires chronic delivery, and thus presents a chronic cancer risk, and even then has a small, inconsistent effect on telomere length, with no detectable effect on telomere length at all in about half of patients.

Accordingly, in some embodiments of the instant methods, the expression of telomerase reverse transcriptase activity, i.e., the half-life of telomerase activity, lasts no longer than 48 hours, no longer than 36 hours, no longer than 24 hours, no longer than 18 hours, no longer than 12 hours, no longer than 8 hours, no longer than 4 hours, or even shorter times. In some embodiments, the expression of telomerase reverse transcriptase activity lasts at least 2 hours, at least 4 hours, at least 8 hours, at least 12 hours, at least 18 hours, at least 24 hours, at least 36 hours, at least 48 hours, or even longer times.

In some embodiments of the instant methods, the transient expression is independent of cell cycle.

As noted above, the transient expression of telomerase reverse transcriptase results in the extension of shortened telomeres in treated cells. Telomere length can be readily measured using techniques such as terminal restriction fragment (TRF) length analysis, qPCR, MMqPCR, and Q-FISH, as would be understood by one of ordinary skill in the art. In some embodiments, the instant methods increase telomere length in treated cells by at least 0.1 kb, at least 0.2 kb, at least 0.3 kb, at least 0.4 kb, at least 0.5 kb, at least 1 kb, at least 2 kb, at least 3 kb, at least 4 kb, at least 5 kb, or even more.

One of the advantages of the instant compounds, compositions, and methods, is the rapidity of extension of telomeres achieved by these techniques. The techniques allow treatments to be brief and thus safe because the normal protective telomere shortening mechanism remains intact for most of the time. Treatment with the compounds and compositions disclosed herein result in delivery of tens or hundreds of copies of TERT modRNA per cell as measured by absolute RT-qPCR, which is substantially more than the average number of copies of endogenous TERT mRNA found even in cells with high telomerase activity. Typically such cells have less than one copy of TERT mRNA per cell. Thus the treatments transiently introduce a large number of copies of modRNA encoding TERT to a cell resulting in rapid telomere extension. Without intending to be bound by theory, the large number of copies of modRNA encoding TERT may transiently overwhelm the inhibitory regulatory mechanisms that normally prevent TERT, and other methods of telomere extension, from extending telomeres as rapidly as the compounds, compositions, and methods disclosed herein.

The transient expression of telomerase reverse transcriptase also results in an increased replicative capacity in treated cells. Increased replicative capacity is readily monitored in cells that are approaching replicative senescence by measuring additional population doublings in such cells. Senescent cells are not stimulated to divide by passage in culture or treatment with serum. Senescent cells are further often characterized by the expression of pH-dependent (3-galactosidase activity, expression of cell cycle inhibitors p53 and p19, and other altered patterns of gene expression, and an enlarged cell size.

Accordingly, in some embodiments, the instant treatment methods increase the number of population doublings by at least one, two, four, or even more population doublings. In some embodiments, the treatment methods increase the number of population doublings by at least 5, 10, 15, 20, or even more population doublings.

In some of the instant method embodiments, the disclosed compounds or compositions are administered to the animal RNA immune-responsive cell by electroporation. In specific embodiments, a disclosed compound is administered to the animal RNA immune-responsive cell by electroporation in the absence of a delivery vehicle. In other specific embodiments a disclosed compound and a telomerase RNA component are administered to the animal RNA immune-responsive cell by electroporation.

Kits

Also disclosed are ready-to-use kits for use in extending telomeres in a mammalian RNA immune-responsive cell. The kits comprise any of the above-described compounds or compositions, together with instructions for their use. In some embodiments, the kits further comprise packaging materials. In some embodiments, the packaging materials are air-tight. In these embodiments, the packaging materials may optionally be filled with an inert gas, such as, for example, nitrogen, argon, or the like. In some embodiments, the packaging materials comprise a metal foil container, such as, for example, a sealed aluminum pouch or the like. Such packaging materials are well known by those of ordinary skill in the art.

In some embodiments, the kit may further comprise a desiccant, a culture medium, an RNase inhibitor, or other such components. In some embodiments, the kit may further comprise a combination of more than one of these additional components. In some kit embodiments, the composition of the kit is sterile.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: Rejuvenation of Human Chondrocytes by Transient Expression of hTERT

Human chondrocytes were acquired from PromoCell (Germany). Current donors are listed in Table 6.

TABLE 6
Current Donors

| ID | Race | Sex | Age | Origin |
|---|---|---|---|---|
| 3060501.3 | caucasian | female | 49 | femoral head |
| 3061805.2 | caucasian | female | 77 | femoral head |
| 1080202.2 | caucasian | male | 67 | femoral head |

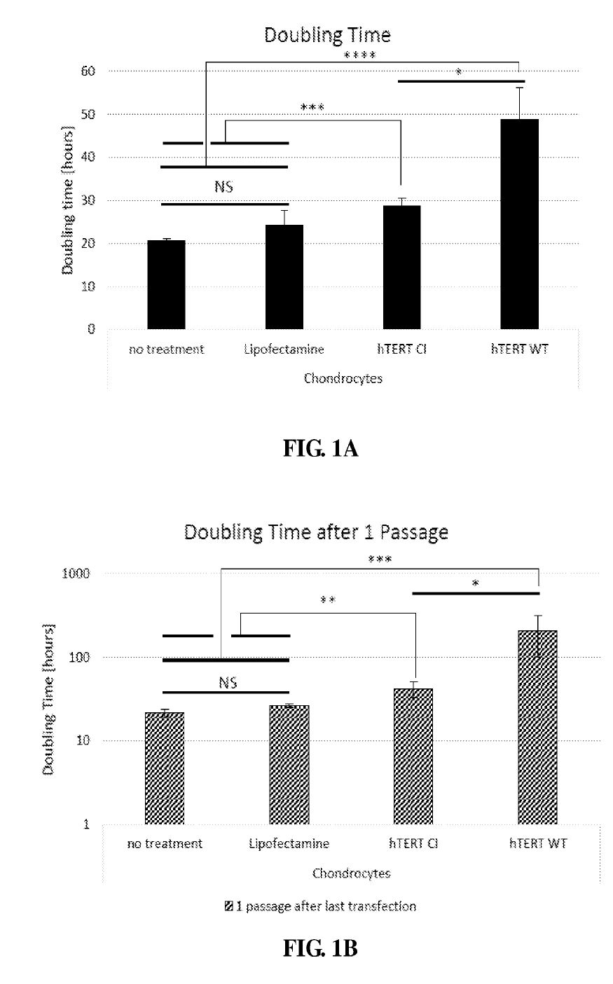

FIGS. 1A and 1B are bar graphs showing chondrocyte doubling times (in hours) after no treatment, treatment with lipofectamine, treatment with hTERT CI, or treatment with hTERT WT. Results are shown after transfection (FIG. 1A, n=6) or 1 passage after transfection (FIG. 1B, n=3).

As shown in Table 7, hTERT treatment alone does not lead to telomere extension. Telomere length in chondrocytes was measured using relative quantification for the telomeric repeat. No extension of telomeres was detected after treatment with hTERT WT mmRNA.

TABLE 7
Telomere Extension after hTERT treatment
P6 Chondrocytes double treatment 1.5 μg/mL mmRNA(n = 4)

| | | |
|---|---|---|
| AVERAGE | STDEV | p-value (vs. UN) |

| | | | |
|---|---|---|---|
| Lipofectamine | 1.03 | 0.02 | 0.11 |
| hTERTCI | 1.00 | 0.01 | 0.98 |
| hTERT WT | 1.01 | 0.01 | 0.18 |

One possible explanation for the lack of telomere extension is that there is activation of innate immunity in chondrocytes in response to modified messenger RNA (no effect of nGFP seen) or activation of innate immunity by telomerase itself through a non-canonical pathways. There are two possible pathways that could facilitate a pro-inflammatory response of chondrocytes and could lead to cell cycle arrest or cell death. These include activation of NFkB through non-canonical pathway of hTERT, and activation of interferon pathway by mmRNA or hTERT(JAK-STAT).

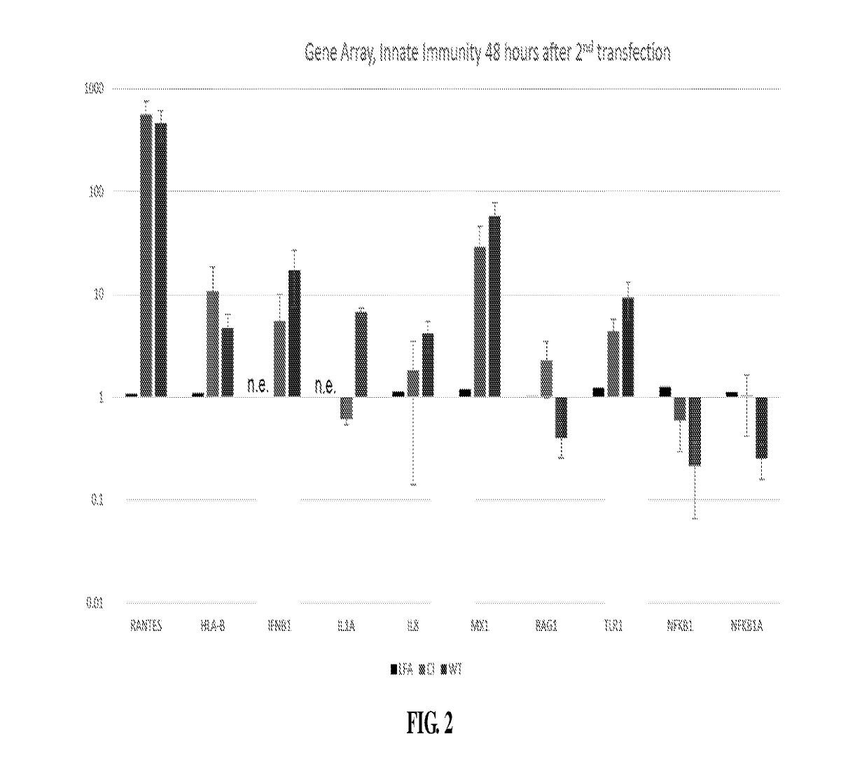

As shown in FIG. 2, hTERT mmRNA activates innate immunity. There are two alternative strategies to reduce activation of innate immunity—inhibition of the NFkB pathway, and inhibition of the interferon pathway.

For the NFkB pathway, the RelA/NFkB p65 [pSer529, pSer536] inhibitor peptide was used (NOVUS) at 10 μM and 50 μM. Both concentrations are lower than the concentration needed to completely suppress NFkB expression.

For the interferon pathway, B18R protein, which is a vaccinia virus-encoded receptor with specificity for type I interferons, was used at a concentration of 200 ng/mL.

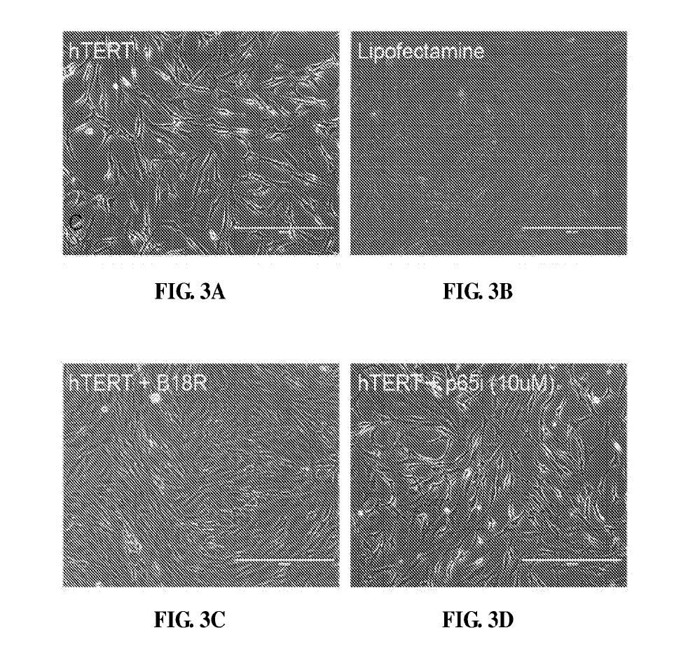

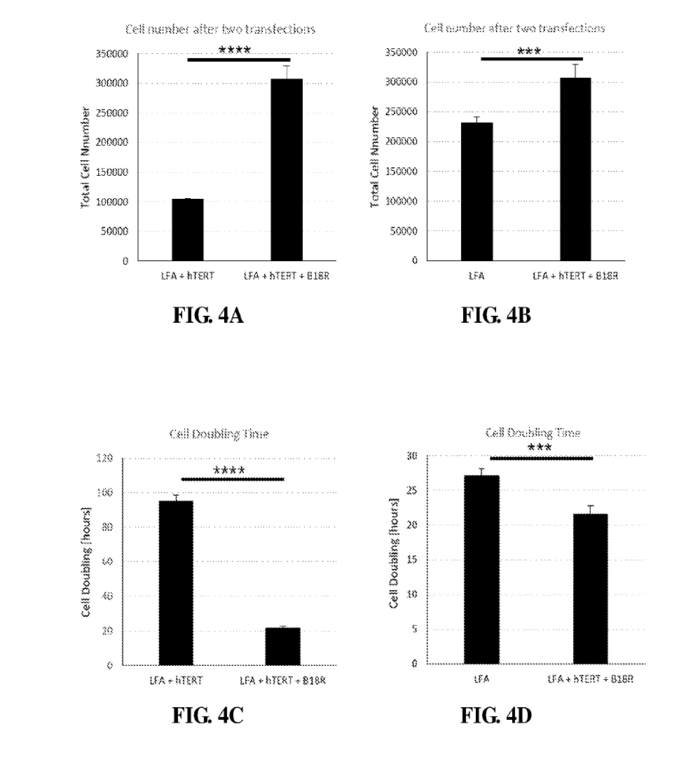

As shown in FIGS. 3A to 3D, reducing NFkB activation and interferon signaling reduces adverse effects of hTERT. In addition, reducing NFkB activation and interferon signaling increased chondrocyte proliferation capacity (FIGS. 4A to 4D).

As shown in Table 8, reducing activation of innate immunity allows telomere elongation by hTERT mmRNA (n=3).

TABLE 8
Telomere elongation by hTERT mmRNA with
reduced activation of innate immunity

| | | | | % increase | |
|---|---|---|---|---|---|
| Intervention | | | T/S-ratio | in TL | p-value |
| LFA | — | — | 0.97 | | — |
| | — | P65i high | 1.01 | 4.0 | 0.27 |
| | — | B18R | 0.99 | 2.7 | 0.36 |
| | hTERT | — | 1.03 | 6.0 | 0.08 |
| | | P65i low | 1.00 | 3.0 | 0.65 |
| | | P65i high | 1.06 | 8.7 | 0.06 |
| | | B18R | 1.05 | 7.8 | 0.02 |
| | | both | 1.08 | 10.8 | 0.04 |

Repeated experiments of hTERT + B18R + p65i treatment (n = 6, two donors): 9.2 ± 3.4% compared to no treatment (p < 0.001)

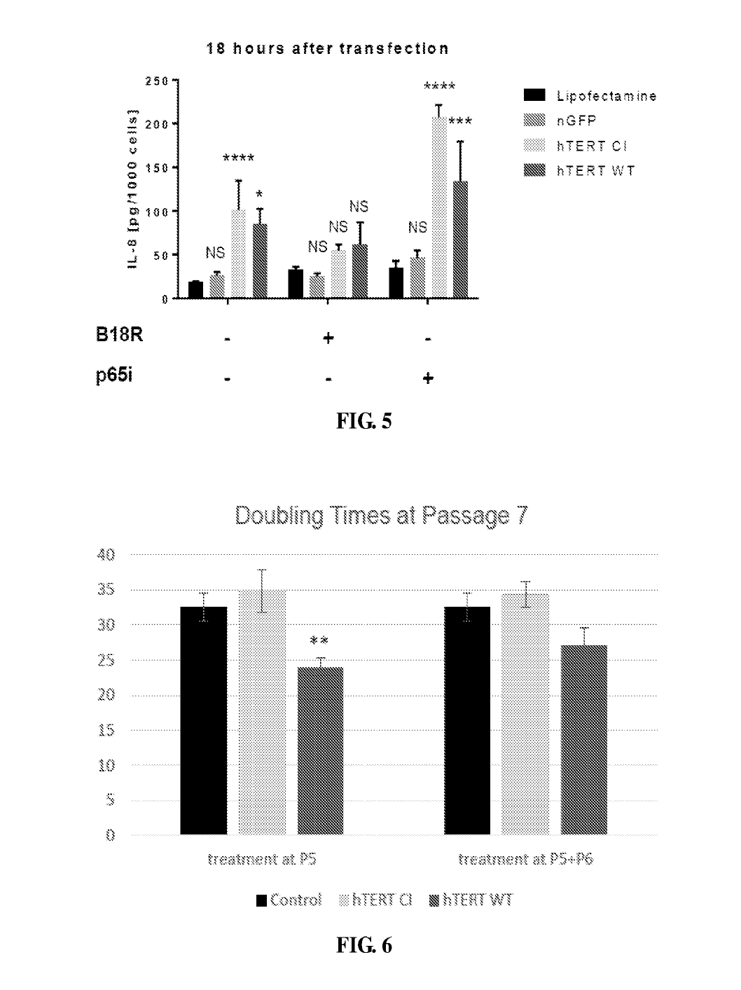

As shown in FIG. 5, B18R decreases IL-8 secretion after hTERT transfection (n=3).

As shown in FIG. 6, double transfection at early passage with an improved protocol increased proliferative capacity. Transfection with hTERT CI and WT was performed in the presence of B18R and p65i (10 μM). For the control, cells were expanded in regular chondrocyte media with use of lipofectamine at passage 5. Table 9 shows passages when senescence was reached (PD<1.5).

TABLE 9
Passage with senescence reached

| Donor/Condition | Untreated | hTERT CI | hTERT WT |
|---|---|---|---|
| 3061805.21x treatment | Passage 9 | Passage 9 | Passage 11 |
| 3061805.23x treatments | Passage 9 | Passage 9 | Passage 10 |
| 1080202.21x treatment | Passage 9 | Passage 9 | Passage 9 |
| 1080202.23x treatment | Passage 8 | Passage 8 | Passage 9 |

### Example 2: Suppression of Immune Response During TERT Transfection

The data in Example 1 shows that during the transient transfection of chondrocytes with wildtype and catalytic inactive TERT many pro-inflammatory cytokines are secreted. RANTES was identified as the cytokine with the most specific response to the aforementioned transfections. It was not secreted before transfection, it was not secreted upon transfection with the vector, and it was not secreted upon the transfection with nGFP.

When suppressing interferon binding and NFkB activation in chondrocytes during transfection, no RANTES secretion and no adverse effects in Chondrocytes were observed.

Therefore RANTES was used as a test screening marker when using clinically available suppressors of immune response. RANTES may be easily measured via a regular ELISA assay. Initial experiments were performed with a magnetic bead based ELISA assay that allows measurement up to 64 cytokines in one sample at once [MILLIPLEX LUMINEX by EMD Millipore].

Chondrocytes were plated in a 96-well plate that would allow fluorescent imaging after fixing the cells. Cells are plated at 10,000 cells/cm² and cultured for 48 hours. In this assay RNATES secretion into the cell culture media was measured. Every cytokine has its own secretion dynamic upon stimulation. The cells for each time point were plated in separate plates. After the collection at each time point, cells were fixed, stained with DAPI and an automated nuclear cell count was performed. This is done to normalize the cytokine secretion to the amount of cells that were present at each given time point.

After culturing the cells for 48 hours, two hours prior to transfection each component was added to the respective wells in triplicates. Then at time point of transfection the media from the 0 time point plate were collected to have the baseline cytokine levels before transfection. Then at 18 hours the media was collected from the second plate and cell count is acquired as described above.

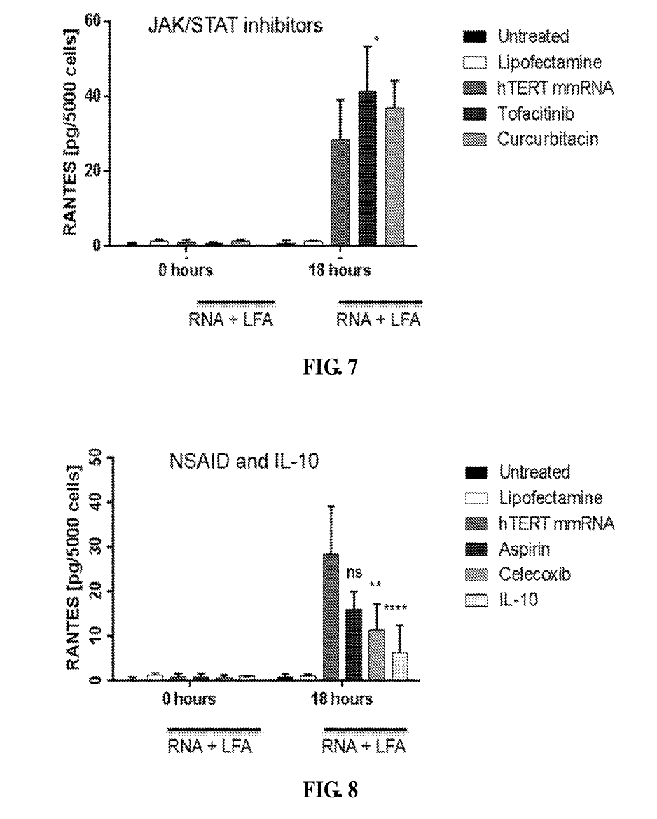

FIGS. 7 and 8 show results of anti-inflammatory agents tested, including JAK/STAT inhibitors (FIG. 7) and NSAIDs and IL-10 (FIG. 8).

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method for promoting, enhancing or assisting with the rejuvenation of one or more RNA immune-responsive cells, comprising contacting the RNA immune-responsive cells with a composition comprising an RNA encoding a telomerase reverse transcriptase, and a composition comprising an anti-inflammatory, in amounts effective to improve the function or replicative capacity of the cells.

2. The method of claim 1, wherein the method extends at least one telomere in the one or more RNA immune-responsive cells.

3. The method of claim 1, wherein the RNA immune-responsive cells comprise one or more chondrocytes.

4. The method of claim 3, wherein the chondrocytes are obtained from subjects with cartilage degeneration prior to the contacting step.

5. The method of claim 1, wherein the RNA immune-responsive cells comprise one or more mesenchymal stromal cells (MSC).

6. The method of claim 1, wherein the RNA immune-responsive cells are endothelial cells, myoblasts or T-lymphocytes.

7. The method of claim 1, wherein the RNA immune-responsive cells are in a tissue, organ, or blood, or whole organism.

8. The method of claim 1, wherein the RNA immune-responsive cell is a cell that has upregulated RANTES expression when contacted with a synthetic ribonucleic acid comprising at least one modified nucleoside encoding a telomerase reverse transcriptase.

9. The method of claim 1, wherein the anti-inflammatory comprises an interferon antagonist, an NFκB antagonist, or a combination thereof.

10. The method of claim 9, wherein the interferon antagonist comprises a B18R protein.

11. The method of claim 9, wherein the NFκB antagonist comprises a RelA/NF-kB p65 [p Ser529, p Ser536] Inhibitor Peptide.

12. The method of claim 1, wherein the anti-inflammatory comprises a Jak-Stat inhibitor.

13. The method of claim 12, wherein the Jak-Stat inhibitor comprises Tofacitinib, Curcurbitacin, or a combination thereof.

14. The method of claim 1, wherein the anti-inflammatory comprises a non-steroidal anti-inflammatory drug (NSAID).

15. The method of claim 14, wherein the NSAID is selected from the group consisting of Indomethacin, Ibuprofen, and Celecoxib.

16. The method of claim 1, wherein the anti-inflammatory comprises a steroid.

17. The method of claim 16, wherein the steroid is selected from the group consisting of celastrol, dexamethasone, and prednisone.

18. The method of claim 1, wherein the anti-inflammatory comprises an immunosuppressant.

19. The method of claim 18, wherein the immunosuppressant comprises rapamycin, everolimus, or a combination thereof.

20. The method of claim 1, wherein the anti-inflammatory comprises an anti-inflammatory cytokine.

21. The method of claim 20, wherein the anti-inflammatory cytokine comprises IL-10.

22. The method of claim 1, wherein the anti-inflammatory comprises an analgesic.

23. The method of claim 20, wherein the analgesic comprises aspirin, acetaminophen, or a combination thereof.

24. The method of claim 1, wherein the anti-inflammatory is a naturally occurring anti-inflammatory agent.

25. The method of claim 1, wherein the anti-inflammatory inhibits or prevents upregulated RANTES expression in the cell when contacted with a synthetic ribonucleic acid comprising at least one modified nucleoside encoding a telomerase reverse transcriptase.

26. The method of claim 1, wherein the method further comprises contacting the RNA immune-responsive cell with a composition comprising a synthetic ribonucleic acid and the interferon antagonist.

27. The method of claim 1, wherein method further comprises measuring telomerase activity in the one or more RNA immune-responsive cells prior to the contacting step.

28. The method of claim 27, wherein the one or more RNA immune-responsive cells have at least one shortened telomere prior to the contacting step.

29. The method of claim 1, wherein the telomerase reverse transcriptase is a human telomerase reverse transcriptase.

30. The method of claim 1, wherein the RNA encoding a telomerase reverse transcriptase comprises a 5′ cap, a 5′ untranslated region, a 3′ untranslated region, and a poly-A tail.

31. The method of claim 30, wherein the 5′ cap is nonimmunogenic.

32. The method of claim 30, wherein the 5′ cap has been treated with phosphatase.

33. The method of claim 30, wherein the 5′ untranslated region or the 3′ untranslated region comprise a sequence from a stable mRNA or an mRNA that is efficiently translated.

34. The method of claim 30, wherein the 5′ untranslated region and the 3′ untranslated region both comprise a sequence from a stable mRNA or an mRNA that is efficiently translated.

35. The method of claim 1, wherein the at least one modified nucleoside modulates immunogenicity of the ribonucleic acid.

36. The method of claim 26, wherein the synthetic ribonucleic acid is a purified synthetic ribonucleic acid.

37. The method of claim 36, wherein the synthetic ribonucleic acid is purified to remove immunogenic components.

38. The method of claim 37, wherein the composition comprising the synthetic ribonucleic acid further comprises a telomerase RNA component.

39. The method of claim 38, wherein the telomerase RNA component is a human telomerase RNA component.

40. The method of claim 39, wherein the composition comprising the synthetic ribonucleic acid further comprises a delivery vehicle.

41. The method of claim 40, wherein the delivery vehicle is an exosome, a lipid nanoparticle, a polymeric nanoparticle, a natural or artificial lipoprotein particle, a cationic lipid, a protein, a protein-nucleic acid complex, a liposome, a virosome, or a polymer.

42. The method of claim 41, wherein the delivery vehicle is non-immunogenic.

43. The method of claim 1, wherein the method further comprises the step of measuring the average telomere length in the RNA immune-responsive cells.

44. The method of claim 43, wherein average telomere length in the RNA immune-responsive cell is increased by at least 0.1 kb.

45. The method of claim 26, wherein contacting the RNA immune-responsive cells with the composition comprising the synthetic ribonucleic acid comprises electroporation.

46. The method of claim 1, wherein the composition further comprises a transfection agent.

47. The method of claim 46, wherein the transfection agent comprises a liposome.

48. The method of claim 47, wherein the liposome comprises DOTAP and cholesterol in a 1:1 molar ratio.

49. The method of claim 47, wherein the liposome further comprises protamine.

50. The method of claim 1, further comprising administering a plurality of the rejuvenated RNA immune-responsive cells to a subject in need thereof.

51. A method for promoting, enhancing or assisting with the rejuvenation of cartilage in a subject, comprising administering to the subject a plurality of rejuvenated chondrocyte produced by the method of claim 1.

52. A method for promoting, enhancing or assisting with the rejuvenation of cartilage in a subject, comprising administering to the subject a composition comprising a synthetic ribonucleic acid comprising at least one modified nucleoside encoding a telomerase reverse transcriptase, and a composition comprising an interferon antagonist, in amounts effective to extend at least one telomere in chondrocytes within the cartilage.

53. The method of claim 52, wherein the method comprises administering to the subject a composition comprising the synthetic ribonucleic acid and the interferon antagonist.

54. The method of claim 52, wherein the composition is administered within the joint capsule of the subject.
